(19) United States
(12) Patent Application Publication
(10) Pub. No.: US 2020/0019240 A1
(43) Pub. Date: Jan. 16, 2020

(54) DATA TRANSMISSION METHOD AND DATA TRANSMISSION DEVICE

(71) Applicants: VESTEL ELEKTRONIK SANAYI VE TICARET A.S., Manisa (TR); OZYEGIN UNIVERSITESI, Istanbul (TR)

(72) Inventors: Burhan GULBAHAR, Manisa (TR); Ozgur YILDIZ, Manisa (TR)

(73) Assignees: VESTEL ELEKTRONIK SANAYI VE TICARET A.S., Manisa (TR); OZYEGIN UNIVERSITESI, Istanbul (TR)

(21) Appl. No.: 16/471,712

(22) PCT Filed: Dec. 20, 2016
(86) PCT No.: PCT/EP2016/081998

§ 371 Date: Jun. 20, 2019

(51) Int. Cl. G06F 3/01; A61B 3/113; G02B 27/00

(52) U.S. Cl. CPC G06F 3/013; G02B 2027/0187; G02B 27/0093; A61B 3/113

(57) ABSTRACT

The present disclosure provides a data transmission method for transmitting data. via visible light with a display device that displays an image. The method comprises detecting eye movements of a number of users of the display device, calculating the focus areas of the users in the displayed image, and modulating the displayed image in a modulation area according to the data to be transmitted, wherein the modulation area comprises the image surface without the focus areas of the users. Further, the present disclosure provides a data transmission device.

### CROSS-REFERENCE TO RELATED APPLICATION

This application is the U.S. national phase of PCT Application No. PCT/EP2016/081998 filed on Dec. 20, 2016, the disclosure of which is incorporated in its entirety by reference herein.

### TECHNICAL FIELD

The disclosure relates to a data transmission method and to a data transmission device.

### BACKGROUND

Although applicable to any system that uses data transmission via light, embodiments of the present disclosure will mainly be described in conjunction with data transmission via visible light.

Modern communication systems should provide increasingly high data rates to transmit high bandwidth data, like e.g., HD video data or the like.

In electromagnetic data transmission systems, like e.g., Ethernet or WLAN, a plurality of approaches are used to increase the bandwidth of the respective systems. However, to further increase the possible bandwidth light and especially visible light may be used to transmit data. Data transmission via light however must take into account that humans may detect, i.e., see, modulation artefacts in the light. In such cases acceptance of the light modulation may be low.

To avoid visible modulation artefacts the influence of the modulation on the visible light is usually kept low. This can either be done by using light outside of the visible spectrum to transmit data or by using only slight modulations over visible light that cannot be detected or are not considered as disturbing. This however reduces the possible bandwidth. Data transmission via visible light according to the HiLight standard e.g., only allows a 1% change of the emitted light.

Accordingly, there is a need for an improved modulation of visible light for data transmission.

### SUMMARY

The present disclosure provides a data transmission method and a data transmission device.

A data transmission method for transmitting data via visible light with a display device that displays an image comprises detecting eye movements of a number, e.g., one or more, of users of the display device, calculating the focus areas of the users in the displayed image, and modulating the displayed image in a modulation area according to the data to be transmitted, wherein the modulation area comprises the image surface without the focus areas of the users.

A data transmission device for transmitting data via visible light with a display device that displays an image comprises an eye movement detector configured to detect eye movements of a number of users of the display device, a focus calculator configured to calculate the focus areas of the users in the displayed image, and an image modulator configured to modulate the displayed image in a modulation area according to the data to be transmitted, wherein the modulation area comprises the image surface without the focus areas of the users.

The present disclosure is based on the finding that the human eye can only focus to a specific focus point at a time and that a human can only see sharply in a very small area around that focus point.

The present disclosure now uses this knowledge and provides a method for data transmission via visible light that can be used with any kind of display devices, like e.g., TV sets, video projectors, digital signage signs, tablet PCs or smartphones or the like.

The present disclosure is based on detecting the movements of the eyes of the users of the display device with an optical sensor, e.g., with a camera, that can e.g., be embedded in a TV or a tablet PC or the like. However, dedicated optical sensors can be provided to detect the users' eye movements. It is understood that any adequate method for detecting the eye movements can be used. For example light, typically infrared, is reflected from the eye and can be sensed by the optical sensor. The information can then be analyzed to extract eye movement or rotation information from changes in reflections. Video-based eye tracking can also use a corneal reflection and a center of the pupil as features to track over time.

Based on the detected eye movements the focus areas are calculated for the single users. The focus areas refer to the areas on the displayed image which the user can perceive sharply. These areas with human eyes are usually relatively small compared to the image width.

Finally, the displayed image is modulated according to the detected focus areas. Modulating in this context refers to modulating the image data of the displayed image in the modulation area. The modulation area only comprises regions of the displayed image that are not part of any of the detected focus areas.

The present disclosure uses only sections of the displayed image that are not perceived sharply by the users of the display device to transmit the data. Therefore, the modifications to that section of the displayed image can be drastically increased as compared to modulating the whole image based on the assumption that the whole image could or would be viewed at any given time by a user.

The modulated picture or video may then e.g., be recorded via a camera of an electronic device for regeneration of the original data or message. Such an electronic device can e.g., be a smartphone a tablet pc or the like. The mobile device may therefore comprise the respective demodulation functions or blocks, that are required to perform the demodulation of the modulated image or video.

The present disclosure therefore provides a data communication system to transmit data from a display to any camera enabled device.

Further embodiments of the present disclosure are subject of the further subclaims and of the following description, referring to the drawings.

In an embodiment, detecting the eye movements of the users can comprise recording images of the users and detecting the eyes of the users in the recorded images. Recording an image can e.g., be performed by any type of camera. Such a camera can e.g., be embedded in the frame of a TV or in a smartphone or tablet. Alternatively, an additional camera can be positioned independently of the display device to track the users' eye movements. The detection can e.g., be performed by an eye detector component. The eye detector component can e.g., be a software component that is executed on a processor e.g., in the camera or any other processor of the data transmission device.

In an embodiment, calculating the focus areas can comprise calculating the focus point of the users and calculating areas that correspond to a viewing angle of 0° to 10°, especially 8°, 6°, 4° or 2° around the respective viewing points. The viewing angle of the human eye that defines the focus area, i.e., the section of the complete view that the person perceives sharply is very limited. By adapting the focus area to this very limited section of the complete view, a larger section of the image can be provided for modulation of data.

In an embodiment, calculating the focus areas can comprise calculating at least two areas around the respective viewing points with different viewing angels, or radiuses, wherein the viewing angle or radius of the respective area defines the amount of modulation of the respective area. Stepwise calculating areas in this context refers to calculating more than a single area around the respective focus point, wherein the areas comprise different radius ranges and are arranged like rings around the focus point, as e.g., the rings of a shooting target disk or the like. The distance of the respective ring can e.g., define the amount of modification to the respective area that is allowed to modulate data into the image. The amount can e.g., be defined as a percentage of modification e.g., of a color or brightness value. It is understood, that the areas can also be elliptical with different radiuses.

In an embodiment, the data transmission method can comprise calculating the modulation area by masking out in the displayed image the focus areas of the users. Masking is a very efficient way to separate the areas of the image that can be used for modulation from the areas of the image that cannot be used for modulation. Such a mask can e.g., be provided like the “alpha” layer in image manipulation programs or like the alpha information in RGBA image data. However, any other masking type could be used. The information in the mask can also define a grade or amount of modulation for the respective section.

In an embodiment, modulating can comprise modifying color and/or intensity of the displayed image according to the data to be transmitted in the modulation area. Modifying color and/or intensity of the single pixels or areas of the image is an effective way of providing and transmitting information via the visible light emitted via the displayed image. It is understood that such modulations can e.g., comprise embedding information as random background noise in the image, color shifting, or modifying any other characteristic property of the image.

It is for example possible that two modulations, e.g., color and brightness based modulation, are both used at the same time.

In an embodiment, modulating can comprise modulating the content of the displayed image such that the original image data is modified between 20% to 90%, especially 40%, 60% or 80%. The amount of modification between 20% and 90% refers e.g., to the change in color or brightness of the respective areas of the displayed image. That means that in the modulation area e.g., the brightness can change up to 90% of the original brightness value. The brightness of respective areas or pixels can e.g., be lowered or increased to modulate the respective data into the displayed image. In addition, or as an alternative, a color modulation can be applied, where the color values of the respective images are modified up to 90%. This change can either refer to a single color or alpha channel or to the combined color information. The color basis for the modulation can be in any adequate color scheme, e.g., RGB, YMCK or the like. The color scheme can e.g., be the color scheme used by the display device.

### BRIEF DESCRIPTION OF THE DRAWINGS

For a more complete understanding of the present disclosure and advantages thereof, reference is now made to the following description taken in conjunction with the accompanying drawings. The disclosure is explained in more detail below using exemplary embodiments, which are specified in the schematic figures of the drawings, in which:

FIG. 1 shows a flow diagram of an embodiment of a method according to the present disclosure;

FIG. 2 shows a flow diagram of another embodiment of a method according to the present disclosure;

FIG. 3 shows a block diagram of an embodiment of a device according to the present disclosure; and

FIG. 4 shows a block diagram of another embodiment of a device according to the present disclosure.

In the figures like reference signs denote like elements unless stated otherwise.

### DETAILED DESCRIPTION

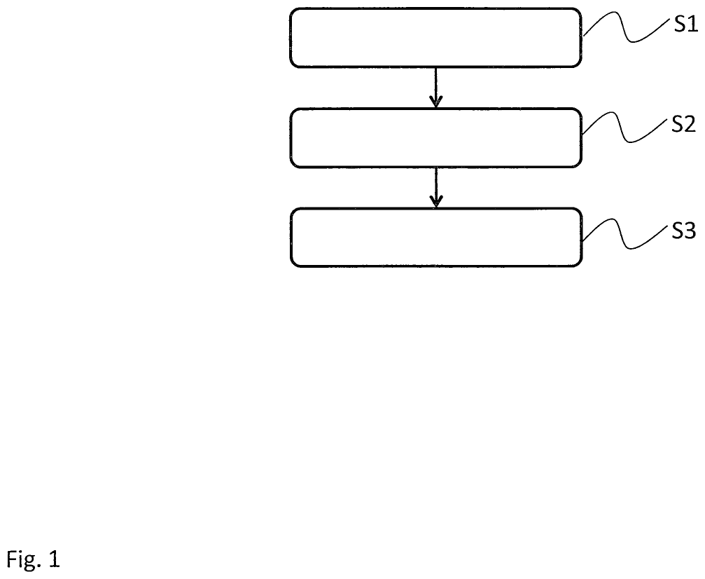

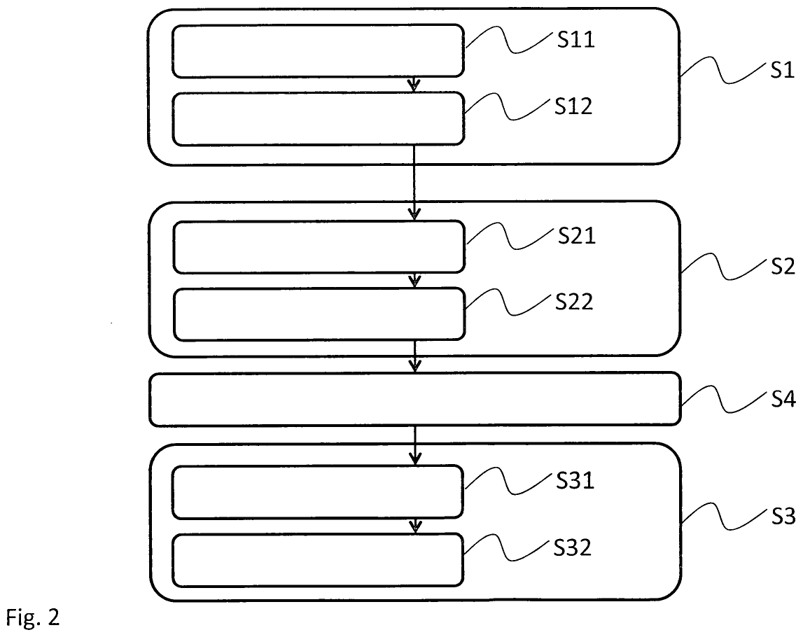

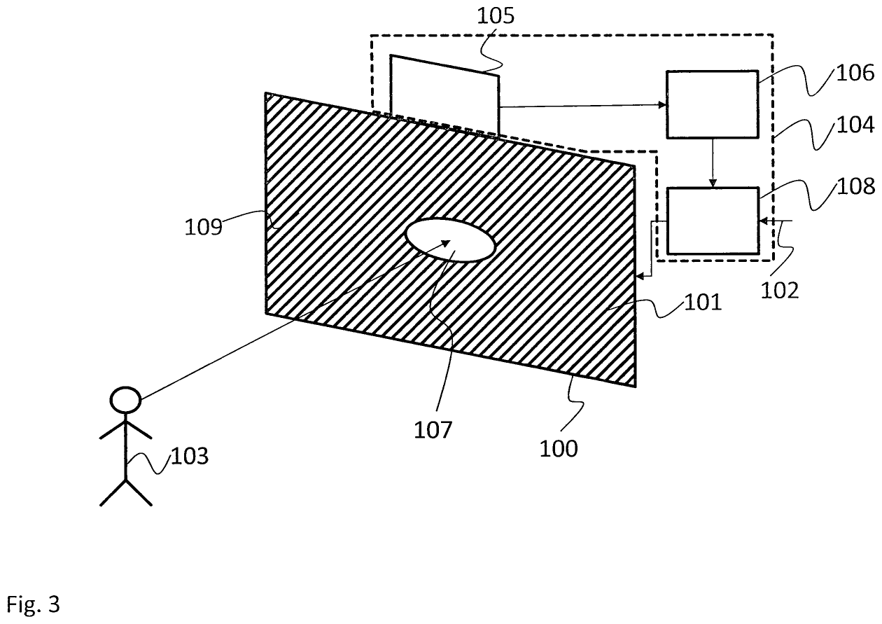

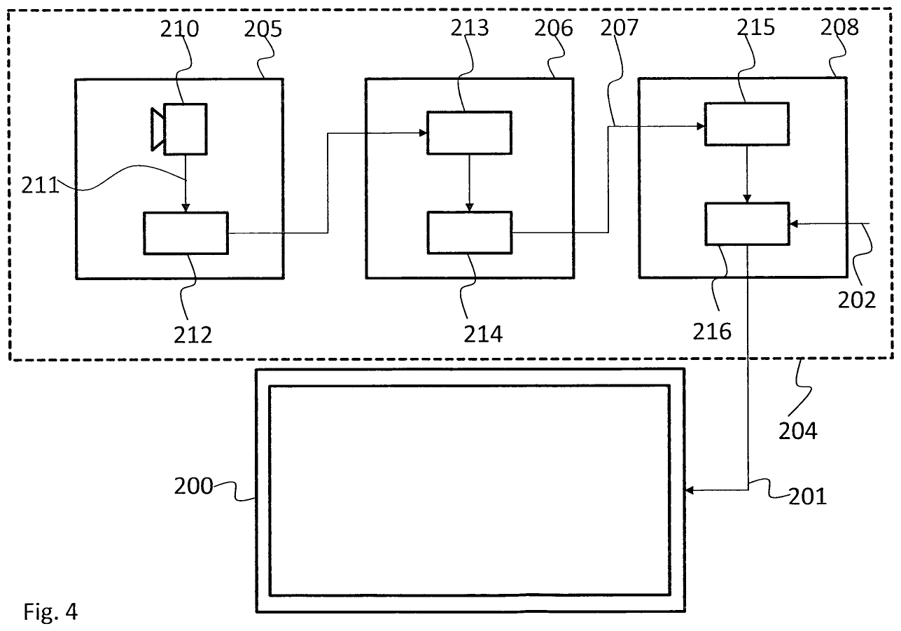

The reference signs of the device elements used with FIGS. 3 and 4 are also used in the description of the method in FIGS. 1 and 2 for sake of clarity.

FIG. 1 shows a flow diagram of a data transmission method for transmitting data 102 via visible light with a display device 100, 200 that displays an image 101.

The method comprises detecting S1 eye movements of a number of users 103 of the display device 100, 200, The eye movements of any number of users 103, e.g., one or more can be tracked or detected in this step S1.

After detecting the eye movements of the users 103, the focus areas 107, 207 of the users 103 in the displayed image 101 are calculated 32. The focus areas 107, 207 refer to the focus point, at which the respective user 103 is looking and a predefined area around that focus point.

Finally, the displayed image 101 is modulated in a modulation area 109, 209 according to the data 102 to be transmitted. The modulation area 109, 209 comprises the image surface without the focus areas 107, 207 of the users 103. That means that the modulation area 109, 209 decreases with the number of focus areas 107, 207. However, it is possible that e.g., different users 103 are looking at the same focus point and that they therefore have the same or overlapping focus area 107, 207.

FIG. 2 shows a flow diagram of another data transmission method that is based on the data transmission method of FIG. 1.

In the method of FIG. 2 detecting S1 the eye movements of the users 103 comprises recording S11 images 211 of the users 103 and detecting S12 the eyes of the users 103 in the recorded images 211.

Calculating 32 the focus areas 107, 207 comprises calculating 321 the focus point of the respective user 103 e.g., based on the position and orientation of the eyes as detected in the images 211.

Based on the information about the focus point of the users 103, the focus area 107, 207 for every single user can he calculated S22. The focus area 107, 207 can e.g. correspond to a viewing angle of 0° to 10° around the respective viewing point, especially 8°, 6°, 4° or 2°.

Calculating S22 the focus areas 107, 207 can also comprise calculating at least two areas around the respective viewing points with different viewing angels. The viewing angle of the respective area can then define the amount of modulation of the respective area. That means that the distance to the focus point defines the amount of modulation of the respective area. The farther away a focus area is from the focus point the more modulation is allowed in the respective area. The centermost focus area usually will comprise no modulation.

The modulation area 109. 209 can e.g., be defined by masking out in the displayed image 101 the focus areas 107, 207 of the users 103.

Modulating S3 can then comprise modifying the color 331 and/or modifying the intensity S32 of the displayed image 101 according to the data 102 to be transmitted in the modulation area 109, 209. Although in FIG. 2 modifying the color 331 and modifying the intensity 332 is shown, it is understood that only one type of modification is also possible. Further, any other type of visible light modulation scheme can he used. When modulating the image data in the modulation area 109, 209 the content of the displayed image 101 can be modulated such that the original image data is modified between 20% to 90%, especially 40%, 60% or 80%.

FIG. 3 shows a block diagram of a data transmission device 104 for transmitting data 102 via visible light. The data transmission device 104 is arranged together with a TV that displays an image 101. It is understood that the data transmission device 104 can be arranged with or in any other type of display device, like e.g., a smartphone, a tablet PC, a computer or a notebook.

The data transmission device 104 comprises an eye movement detector 105 that is coupled to a focus calculator 106, which is coupled to an image modulator 108.

The eye movement detector 105 detects eye movements of a number of users 103 of the display device 100. When the eye movements of the users 103 are detected, a focus calculator 106 calculates the focus areas 107 of the users 103 in the displayed image 101 based on the detected eye movements. In FIG. 3 only one user 103 is shown. It is understood however that any number of users 103 can be present.

The focus calculator 106 can e.g., determine the orientation of the users 103 gaze and determine the intersection of the direction of the gaze and the displayed image 101. The focus areas 107 define for an image modulator 108, which sections of the displayed image 101 may be used for modulation according to the data 102. The focus calculator 106 can e.g., calculate the modulation area 109 by masking out in the displayed image 101 the focus areas 107 of the users 103.

Usually the modulation area 109 will therefore comprise the image surface or area without the focus areas 107 of the users 103.

The image modulator 108 therefore modulates the displayed image 101 in a modulation area 109 according to the data 102 to be transmitted.

For modulating the data 102 into the image 101 the image modulator 108, can e.g., modify color and/or intensity of the displayed image 101 in the modulation area 109 according to the data 102.

Further, to provide the modulation such that a user does not perceive any disturbance, the image modulator 108 can modulate the content of the displayed image 101 according to a predefined modulation grade, e.g., such that the original image data is modified between 20% to 90%, especially 40%, 60% or 80%.

FIG. 4 shows a block diagram of another data transmission device 204 according to the present disclosure. The data transmission device 204 is based on the data transmission device 104 of FIG. 1 and comprises additional components.

The eye movement detector 205 for example comprises a camera 210 that is coupled to an eye detector component 212. The eye detector component 212 is coupled to a focus point calculation unit 213 that is coupled to a focus area calculation unit 214. The focus area calculation unit 214 is coupled to a modulation area determination unit 215, which is coupled to a modulator 216.

The camera 210 records images 211 of the users and provides the recorded images 211 to the eye detector component 212 that detects the eyes of the users in the recorded images 211.

Based on the detected eyes of the users in the recorded images 211 the calculation function 213 calculates the focus point of the respective users. The focus area calculation function 214 then calculates the focus area 207. The focus area calculation function 214 can e.g., calculate the focus area 207 such that it corresponds to a viewing angle of 0° to 10°, especially 8°, 6°, 4° or 2° around the respective viewing point. The focus area calculation function 213 can also be configured to calculate at least two areas around the respective viewing points with different viewing angels. In such an embodiment the viewing angle of the respective area defines the amount of modulation of the respective area.

With the information about the focus areas 207 the modulation area determination unit 215 of the image modulator 208 determines the modulation areas, i.e., the section of the image 201 that can be used for modulation of the data 202. The modulation area determination unit 215 can also determine a plurality of modulation areas with different amounts of modulation, e.g., for use with the ring-like arranged focus areas, as described above.

Finally, the modulator 216 modulates the data 202 into the image 201 at the modulation areas and forwards the image 201 to the display device 200.

It is understood, that the elements of the data transmission device 104, 204, e.g., the eye movement detector 105, 205, the focus calculator 106, 206 or the image modulator 108, 208 or any of their subordinate elements can be provided as separate or dedicated entities. It is also understood that any of these elements can also be provided e.g., as a program component and can be executed in a processor of the device, e.g., the TV set of tablet PC, in which the data transmission device 104, 204 is provided.

Although specific embodiments have been illustrated and described herein, it will be appreciated by those of ordinary skill in the art that a variety of alternate and/or equivalent implementations exist. It should be appreciated that the exemplary embodiment or exemplary embodiments are only examples, and are not intended to limit the scope, applicability, or configuration in any way. Rather, the foregoing summary and detailed description will provide those skilled in the art with a convenient road map for implementing at least one exemplary embodiment, it being understood that various changes may be made in the function and arrangement of elements described in an exemplary embodiment without departing from the scope as set forth in the appended claims and their legal equivalents, Generally, this application is intended to cover any adaptations or variations of the specific embodiments discussed herein.

The present disclosure provides a data transmission method for transmitting data (102) via visible light with a display device (100, 200) that displays an image (101), the method comprising detecting (S1) eye movements of a number of users (103) of the display device (100, 200), calculating (S2) the focus areas (107, 207) of the users (103) in the displayed image (101), and modulating (53) the displayed image (101) in a modulation area (109, 209) according to the data (102) to be transmitted, wherein the modulation area (109, 209) comprises the image surface without the focus areas (107, 207) of the users (103). Further, the present disclosure provides a respective method.

### LIST OF REFERENCE SIGNS

100, 200 display device
101, 201 image
102, 202 data
103 user
104, 204 data transmission device
105, 205 eye movement detector
106, 206 focus calculator
107, 207 focus areas
108, 208 image modulator
109, 209 modulation area
210 camera
211 images
212 eye detector component
213 focus point calculation function
214 focus area calculation function
215 modulation area determination unit
216 modulator
217 image
S1-S4 method steps
S11, S12 method steps
321, 322 method steps
331, 332 method steps

1-14. (canceled)

15. A data transmission method for transmitting data via visible light with a display device that displays an image to an electronic device, the method comprising:
detecting eye movements of a number of users of the display device;
calculating focus areas of the users in the displayed image based on the detected eye movements, wherein calculating the focus areas comprises calculating a focus point of a respective user and calculating the focus area that corresponds to a viewing angle of 0° to 10° around a respective viewing point; and
modulating the displayed image in a modulation area according to the data to be transmitted, wherein the modulation area comprises an image surface without the focus areas of the users.

16. The data transmission method according to claim 15, wherein detecting the eye movements of the users comprises recording images of the users and detecting eyes of the users in the recorded images.

17. The data transmission method according to claim 15, wherein calculating the focus areas comprises calculating at least two areas around the respective viewing points with different viewing angles, wherein the viewing angle of the respective area defines the amount of modulation of the respective area.

18. The data transmission method according to claim 15 further comprising calculating the modulation area by masking out in the displayed image the focus areas of the users.

19. The data transmission method according to claim 15, wherein modulating comprises modifying color and/or modifying intensity of the displayed image according to the data to he transmitted in the modulation area.

20. The data transmission method according to claim 15, wherein modulating comprises modulating content of the displayed image such that original image data is modified between 20% to 90%, especially 40%, 60% or 80%.

21. A data transmission device for transmitting data via visible light with a display device that displays an image to an electronic device, the device comprising:
an eye movement detector configured to detect eye movements of a number of users of the display device;
a focus calculator configured to calculate focus areas of the users in the displayed image based on the detected eye movements, wherein the focus calculator comprises a calculation function configured to calculate a focus point of a respective user and calculate the focus area that corresponds to a viewing angle of 0° to 10° around a respective viewing point; and
an image modulator configured to modulate the displayed image in a modulation area according to the data to be transmitted, wherein the modulation area comprises an image surface without the focus areas of the users.

22. The data transmission device according to claim 21, wherein the eye movement detector comprises a camera configured to record images of the users and an eye detector component configured to detect eyes of the users in the recorded images.

23. The data transmission device according to claim 21, wherein the calculation function is configured to calculate at least two areas around the respective viewing points with different viewing angles, wherein the viewing angle of the respective area defines the amount of modulation of the respective area.

24. The data transmission device according to claim 21, wherein the focus calculator is configured to calculate the modulation area by masking out in the displayed image the focus areas of the users.

25. The data transmission device according to claim 21, wherein the image modulator is configured to modify color and/or intensity of the displayed image according to the data to be transmitted in the modulation area.

26. The data transmission device according to claim 21, wherein the image modulator is configured to modulate content of the displayed image such that original image data is modified between 20% to 90%, especially 40%, 60% or 80%.
